(19) United States
(12) Patent Application Publication
(10) Pub. No.: US 2010/0105695 A1
(43) Pub. Date: Apr. 29, 2010

(54) METHOD FOR ENHANCING INSULIN SECRETION

(75) Inventors: Arvinder Dhalla, Mountain View, CA (US); Luiz Belardinelli, Palo Alto, CA (US); John Shryock, East Palo Alto, CA (US); Kwan Leung, Palo Alto, CA (US); Dewan Zeng, Palo Alto, CA (US)

Correspondence Address: CV THERAPEUTICS, INC.;Gilead Palo Alto, Inc., 333 Lakeside Drive, Foster City, CA, 94404, US

(73) Assignee: Gilead Palo Alto, Inc., Foster City, CA (US)

(21) Appl. No.: 12/651,891

(22) Filed: Jan. 4, 2010

Related U.S. Application Data
(63) Continuation of application No. 12/101,669, filed on Apr. 11, 2008.
(60) Provisional application No. 60/911,457, filed on Apr. 12, 2007.
(60) Provisional application No. 60/977,009, filed on Oct. 2, 2007.
(60) Provisional application No. 61/026,223, filed on Feb. 5, 2008.

(51) Int. Cl. A61K 31/495; A61P 3/10

(52) U.S. Cl. 514/252.12

(57) ABSTRACT

The invention is directed to methods for enhancing endogenous insulin levels in a patient in need thereof which method comprises administering to the patient an insulin secretion-enhancing amount of racemic ranolazine or the R- or S-enantiomer of ranolazine. It is also directed to methods of treatment comprising racemic ranolazine or the R- or S-enantiomer of ranolazine for enhancing endogenous insulin levels in a patient in need thereof. It is also directed to a composition comprising an insulin secretion-enhancing amount of racemic ranolazine or the R- or S-enantiomer of ranolazine and at least one anti-diabetic agent.

This application claims priority to U.S. Provisional Patent Application Ser. No. 60/911,457, filed Apr. 12, 2007, U.S. Provisional Patent Application Ser. No. 60/977,009, filed Oct. 2, 2007, and U.S. Provisional Patent Application Ser. No. 61/026,223, filed Feb. 5, 2008, the entirety of which are incorporated herein by reference.

### FIELD OF THE INVENTION

The present invention relates to methods for enhancing endogenous insulin levels in a patient in need thereof which method comprises administering to the patient an insulin secretion-enhancing amount of ranolazine (racemate or (±)) or the R- or S-enantiomer of ranolazine. It also relates to methods of treatment and compositions comprising ranolazine or the R- or S-enantiomer of ranolazine for enhancing endogenous insulin levels in a patient in need thereof.

### DESCRIPTION OF THE ART

U.S. Pat. No. 4,567,264, the specification of which is incorporated herein by reference in its entirety, discloses ranolazine, (±)-N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, ranolazine is represented by the formula:

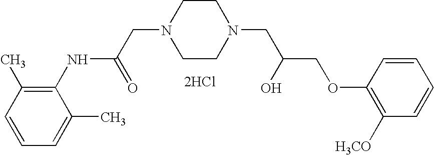

This patent also discloses intravenous (IV) formulations of dihydrochloride ranolazine further comprising propylene glycol, polyethylene glycol 400, Tween 80 and 0.9% saline.

U.S. Pat. No. 5,506,229, which is incorporated herein by reference in its entirety, discloses the use of ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of ranolazine and microcrystalline cellulose coated with release controlling polymers. This patent also discloses IV ranolazine formulations which at the low end comprise 5 mg ranolazine per milliliter of an IV solution containing about 5% by weight dextrose. And at the high end, there is disclosed an IV solution containing 200 mg ranolazine per milliliter of an IV solution containing about 4% by weight dextrose.

The presently preferred route of administration for ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the ranolazine is present in 35-50%, preferably 40-45% ranolazine. In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

### BACKGROUND

Insulin, which is secreted by beta cells of the pancreas, is a necessary hormone which lowers the concentration of glucose in the blood by stimulating the uptake and metabolism, of glucose by muscle and adipose tissue. Insulin stimulates the storage of glucose in the liver as glycogen, and in adipose tissue as triglycerides. Insulin also promotes the utilization of glucose in muscle for energy. Thus, insufficient insulin levels in the blood, or decreased sensitivity to insulin, can give rise to excessively high levels of glucose in the blood.

Carbohydrates (or sugars) are absorbed from the intestines into the bloodstream after a meal. Insulin is then secreted by the pancreas in response to this increase in blood sugar. Most cells of the body have insulin receptors which bind the insulin in the circulation. When a cell has insulin attached to the receptors on its surface, glucose transporters designed to absorb glucose (sugar) from the blood stream are activated. Without insulin, one can consume tremendous amounts of food and actually be in a state of starvation since many of body cells cannot access the calories contained in the glucose without the action of insulin.

Diabetes mellitus is a disorder of metabolism characterized by hyperglycemia (abnormally high level of glucose in the blood). There are two major types of diabetes mellitus: 1) Type I, also known as insulin dependent diabetes mellitus and 2) Type II, also known as insulin independent diabetes mellitus. Even though, in general, Type II refers to “insulin independent diabetes mellitus”, there is a subpopulation of Type II diabetic patients that are capable of responding to enhanced levels of insulin but that do not produce enough insulin on their own.

Type I can be caused by a genetic disorder. The origins of Type I are not fully understood, and there are several theories. It is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia when the amount of secreted insulin drops below the level required for euglycemia (normal blood glucose level). Although the exact trigger for this immune response is not known, all of the possible causes still have the same end result: the pancreas produces very little or no insulin.

In type II diabetes mellitus, either the body does not produce enough insulin or the cells become resistant (fail to respond normally) to the action of the insulin. In either case, the glucose stays in the blood instead of getting absorbed and metabolized by cells. This failure to respond may be due to reduced numbers of insulin receptors on the cells, or a dysfunction of signaling pathways within the cells, or both. The beta cells from the pancreas initially compensate for this insulin resistance by increasing their insulin output. Over time, these cells become unable to produce enough insulin to maintain normal glucose levels, which leads to Type II diabetes mellitus.

The inability of the pancreatic beta cells to produce sufficient quantities of insulin creates several problems. Elevated glucose levels in the blood cause damage to nerves and blood vessels, mainly in the feet, hands, kidneys, eyes, and in other parts of the body as well. Other complications include heart disease, cardiovascular disease, including coronary artery disease (CAD), and stroke.

High blood levels of glucose cause the thickening of the capillary basement membrane, which results in the progressive narrowing of vessel lumina. The vasculopathogies give rise to conditions such as diabetic retinopathy, which may result in blindness, coronary heart disease, intercapillary glomerulosclerosis, neuropathy, and ulceration and gangrene of the extremities.

The toxic effects of excess plasma levels of glucose include the glycosylation of cells and tissues. Glycosylated products accumulate in tissues and may eventually form cross-linked proteins, which are termed advanced glycosylation end products. It is possible that non-enzymatic glycosylation is directly responsible for expansion of the vascular matrix and vascular complications of diabetes mellitus. For example, glycosylation of collagen results in excessive cross-linking, resulting in atherosclerotic vessels. Also, the uptake of glycosylated proteins by macrophages stimulates the secretion of pro-inflammatory cytokines by these cells. The cytokines activate or induce degradative and proliferative cascades in mesenchymal and endothelial cells respectively.

Thus, controlling blood glucose is a highly desirable therapeutic goal. One way to achieve this goal is by providing a method to enhance insulin secretion in a patient in need thereof.

U.S. Pat. No. 4,567,264, the specification of which is incorporated herein by reference, discloses the compound, (±)-N-(2,6-dimethylphenyl)-4-[2-hydroxy-3-(2-methoxyphenoxy)-propyl]-1-piperazineacetamide (known as ranolazine), as well as the R- and S-enantiomers thereof. Ranolazine is approved for the treatment of chronic angina and has been found to be an inhibitor of the late sodium current. It has also been found to be useful for the treatment of congestive heart failure and arrhythmia. See, e.g., U.S. Pat. Nos. 6,528,511 and 6,677,342, and U.S. Patent Publication No. 2003/0220344, the specifications of which are incorporated herein by reference.

While the tolerability of ranolazine in diabetic patients was previous disclosed, see U.S. Patent Publication No. 2002/0052377, heretofore, the use of ranolazine in the treatment of diabetes mellitus was primarily directed to the discovery that ranolazine reduced HbA1c levels in patients to which ranolazine was administered. See, e.g., U.S. Patent Publication No. 2004/0063717, the specification of which is incorporated herein by reference.

While this application discusses treatment of all types of diabetes mellitus including Type I and Type II, it has been unexpectedly discovered that ranolazine, particularly its R-enantiomer, enhances insulin secretion and is effective in treating diabetes mellitus in a class of patients that are insulin-responsive and insulin secretion-deficient. It has also been surprisingly discovered that the R-enantiomer of ranolazine also provides other pharmacokinetic benefits as it provides less inhibition of the CYP2D6 enzyme.

Accordingly, this invention is directed to a method for enhancing endogenous insulin levels in a patient in need thereof which method comprises administering an insulin secretion-enhancing amount of ranolazine or the R- or S-enantiomer of ranolazine. Additionally, the invention is also directed to methods of treating patents suffering from one or more cardiovascular diseases or cardiac disease symptoms which reduces the inter-individual variation among patients, and reduces possible adverse events in patients with poor metabolism all while enhancing endogenous insulin levels, comprising administering the S and/or R-enantiomer of ranolazine to these patients.

### SUMMARY OF THE INVENTION

In one aspect, this invention provides a method for enhancing endogenous insulin levels in a patient in need thereof which method comprises administering an insulin secretion-enhancing amount of ranolazine or the R- or S-enantiomer of ranolazine. Preferably, the patient is insulin-responsive and insulin secretion-deficient.

In another aspect, this invention provides a method for reducing the amount and/or frequency of insulin administration to a patient, which method comprises administering to the patient an insulin secretion-enhancing amount of ranolazine or the R- or S-enantiomer of ranolazine.

In another aspect, this invention provides a method for reducing the amount and/or frequency of administration of anti-diabetic agents to a patient, which method comprises administering to the patient an insulin secretion-enhancing amount of ranolazine, or the R- or S-enantiomer of ranolazine.

In another aspect, this invention provides a method for preserving pancreatic beta cell function in a patient, which method comprises administering to the patient an insulin secretion-enhancing amount of ranolazine, or the R- or S-enantiomer of ranolazine.

In another aspect, this invention provides a method for treating a diabetic patient, which method comprises administering to the patient an insulin secretion-enhancing amount of ranolazine together with at least one additional anti-diabetic agent.

In another aspect, this invention provides a composition comprising an insulin secretion-enhancing amount of ranolazine and at least one additional anti-diabetic agent.

In another aspect, this invention provides a method for enhancing endogenous insulin levels in a patient in need thereof which method comprises administering an insulin secretion-enhancing amount of the R-enantiomer of ranolazine.

In another aspect, this invention provides a method for enhancing endogenous insulin levels in a patient in need thereof which method comprises administering an insulin secretion-enhancing amount of the S-enantiomer of ranolazine.

In another aspect, this invention provides a method for reducing the amount and/or frequency of insulin administration to a patient, which method comprises administering to the patient an insulin secretion-enhancing amount of the R-enantiomer of ranolazine.

In another aspect, this invention provides a method for treating an insulin-resistant patient, which method comprises administering to the patient an insulin secretion-enhancing amount of the R-enantiomer of ranolazine together with at least one additional anti-diabetic agent.

In another aspect, this invention provides a composition comprising an insulin secretion-enhancing amount of the R-enantiomer of ranolazine and at least one additional anti-diabetic agent.

This invention is also directed, in part, to the discovery that R-ranolazine is less of a CYP2D6 inhibitor than racemic ranolazine and thus use of R-ranolazine in the treatment of diabetes and/or one or more cardiovascular diseases provides for the potential reduction of adverse events and/or drug-drug interactions caused by possible dysfunction of the CYP2D6 enzyme and/or co-administration of CYP2D6 substrates.

In another aspect, this invention relates to a method for treating a patient suffering from one or more cardiovascular diseases which method reduces adverse events and/or drug-drug interactions, comprising administering the R-enantiomer of ranolazine to these patients.

In another aspect, this invention relates to a method for treating a patient suffering from one or more cardiovascular diseases which method reduces adverse events in the patient, comprising administering the R-enantiomer of ranolazine to these patients.

In another aspect, this invention relates to a method for treating a patient suffering from one or more cardiovascular diseases which method reduces drug-drug interactions in the patient, comprising administering the R-enantiomer of ranolazine to these patients.

In another aspect, this invention relates to a method for treating a patient suffering from one or more cardiovascular diseases wherein the patient is treated with the R-enantiomer of ranolazine without testing the patient to determine if there is a dysfunction of the CYP2D6 enzyme.

In another aspect, this invention relates to a pharmaceutical composition comprising a therapeutically effective amount of the R-enantiomer of ranolazine or a pharmaceutically acceptable salt, ester, prodrug, or hydrate thereof.

This invention is also directed, in part, to the discovery that S-ranolazine is a more potent inhibitor of the beta adrenoceptor receptors than racemic ranolazine and thus S-ranolazine is useful for the reduction of adverse events as smaller doses of S-ranolazine may be therapeutically equivalent to racemic ranolazine.

In another aspect, this invention relates to a method for treating a patient suffering from one or more cardiovascular diseases which method reduces adverse events comprising administering the S-enantiomer of ranolazine to these patients.

In another aspect, this invention relates to a pharmaceutical composition comprising a therapeutically effective amount of the S-enantiomer of ranolazine or a pharmaceutically acceptable salt, ester, prodrug, or hydrate thereof.

In another aspect, this invention relates to a pharmaceutical composition comprising a therapeutically effective non-equal amounts of the R-enantiomer and the S-enantiomer of ranolazine or pharmaceutically acceptable salts, esters, prodrugs, or hydrates thereof.

In still a further aspect, this invention also relates to methods of treating a diabetic patient suffering from one or more cardiovascular diseases which method reduces adverse events comprising administering a therapeutically effective amount of the R-enantiomer of ranolazine in an amount that is different that the amount of the S-enantiomer to be administered.

### DESCRIPTION OF THE DRAWINGS

FIG. 1 illustrates the effect of ranolazine on glucose stimulated insulin secretion (GSIS) in rat isolated pancreatic islets.

FIG. 2 illustrates insulin levels during an intravenous glucose tolerance test (IVGTT) performed in normal SD rats. The open squares in FIG. 2 relate to ranolazine while the open circles relate to vehicle.

FIG. 3 illustrates insulin levels for (±) ranolazine, the R-enantiomer of ranolazine, and the S-enantiomer of ranolazine (15 mg/kg) during an intravenous glucose tolerance test (IVGTT) performed in normal SD rats. The closed circles in FIG. 3 relate to vehicle, while the closed triangles relate to the R-enantiomer of ranolazine and the open squares relate to the S-enantiomer of ranolazine.

FIG. 4 illustrates the effect of the R-enantiomer of ranolazine (designated “R-”),

the S-enantiomer of ranolazine (designated “S-”), glucose (designated “G”), and a positive control (designated “GLP1”) on GSIS in human isolated pancreatic islets. The number after the designation indicates the concentration of that compound (millimolar for “G”, and micromolar for “R-” and “S-”).

FIG. 5 illustrates the effect of (±) ranolazine on glucose stimulated insulin secretion (GSIS) in human isolated pancreatic islets.

### DETAILED DESCRIPTION OF THE INVENTION

This invention provides methods for enhancing endogenous insulin levels in a patient, preferably in need thereof which method comprises administering an insulin secretion-enhancing amount of ranolazine (racemate or (±)) or the R- or S-enantiomer of ranolazine. The present invention also provides methods of treatment and compositions comprising ranolazine or the R- or S-enantiomer of ranolazine for enhancing insulin secretion in a patient in need thereof.

### DEFINITIONS

In this specification and in the claims that follow, reference will be made to a number of terms that shall be defined to have the following meanings unless otherwise indicated.

“Ranolazine”, when referred to as Ranexa®, is the compound (±)-N-(2,6-dimethylphenyl)-4-[2-hydroxy-3-(2-methoxyphenoxy)propyl]-1-piperazine-acetamide, ranolazine can also exist as its enantiomers(R)-(+)-N-(2,6-dimethylphenyl)-4-[2-hydroxy-3-(2-methoxyphenoxy)-propyl]-1-piperazineacetamide (also referred to as R-ranolazine), and (S)-(−)-N-(2,6-dimethylphenyl)-4-[2-hydroxy-3-(2-methoxyphenoxy)-propyl]-1-piperazineacetamide (also referred to as S-ranolazine), and their pharmaceutically acceptable salts, and mixtures thereof. Unless otherwise stated the ranolazine plasma concentrations used in the specification and examples refer to ranolazine free base. At pH ˜4, in an aqueous solution titrated with hydrogen chloride, ranolazine will be present in large part as its dihydrochloride salt.

Ranolazine, which is named N-(2,6-dimethylphenyl)-4-[2-hydroxy-3-(2-methoxyphenoxy)propyl]-1-piperazineacetamide {also known as 1-[3-(2-methoxyphenoxy)-2-hydroxypropyl]-4-[(2,6-dimethylphenyl)-aminocarbonylmethyl]-piperazine}, can be present as a racemic mixture, or an enantiomer thereof, or a mixture of enantiomers thereof, or a pharmaceutically acceptable salt thereof. Ranolazine can be prepared as described in U.S. Pat. No. 4,567,264, the specification of which is incorporated herein by reference and is also commercially available. The enantiomers of ranolazine can be obtained using conventional methodologies such as chromatographic separation of racemic ranolazine or de novo synthesis from chiral precursors.

“Bradycardia or bradyarrhythmia reducing effective amount” is an amount of ranolazine that treats the bradycardia or bradyarrhythmia.

“Physiologically acceptable pH” refers to the pH of an intravenous solution which is compatible for delivery into a human patient. Preferably, physiologically acceptable pH's range from about 4 to about 8.5 and preferably from about 4 to 7. Without being limited by any theory, the use of intravenous solutions having a pH of about 4 to 6 are deemed physiologically acceptable as the large volume of blood in the body effectively buffers these intravenous solutions.

“Cardiovascular diseases” or “cardiovascular symptoms” refer to diseases or symptoms exhibited by, for example, heart failure, including congestive heart failure, acute heart failure, ischemia, recurrent ischemia, myocardial infarction, STEMI and NSTEMI, and the like, arrhythmias, angina, including exercise-induced angina, variant angina, stable angina, unstable angina, acute coronary syndrome, NSTEACS, and the like, diabetes, and intermittent claudication. The treatment of such disease states is disclosed in various U.S. patents and patent applications, including U.S. Pat. Nos. 6,503,911 and 6,528,511, U.S. Patent Application Nos. 2003/0220344 and 2004/0063717, the complete disclosures of which are hereby incorporated by reference.

“Topical administration” shall be defined as the delivery of the therapeutic agent to the surface of the wound and adjacent epithelium.

“Parenteral administration” is the systemic delivery of the therapeutic agent via injection to the patient.

“Substrate” refers to a compound that is metabolized by a given enzyme.

“Inhibitor” refers to a compound that “slows down” the metabolism of a substrate. Inhibitors may be classified into strong, moderate and weak categories. Strong inhibitors, for example including bupropion, fluoxetine, paroxetine, and quinidine, can cause a >5-fold increase in the plasma AUC values or more than 80% decrease in clearance. Moderate inhibitors, for example including duloxetine and terbinafine, can cause a >2-fold increase in the plasma AUC values or 50-80% decrease in clearance. Weak inhibitors, for example including amiodarone and cimeti dine, can cause a >1.25-fold but <2-fold increase in the plasma AUG values or 20-50% decrease in clearance.

“Inducer” refers to a compound that “speeds up” the metabolism of a substrate.

“Extensive metabolizer” or EM refers to the group of people who have a normal response to the standard dose of a particular drug.

“Intermediate metabolizer” or IM refers to the group of people who may have the problems of poor metabolizers, though usually not as serious.

“Poor Metabolizer or PM refers to the group of people who have problems processing the standard dose of a drug, because their genes do not produce a functional enzyme. Depending on the type of medication, the drug may not be metabolized rapidly enough and a standard dose may lead to the side effects seen in an overdose. Or, the person may not produce enough enzyme to convert it to its active form and a standard dose may not have any therapeutic effect.

“Ultra metabolizer” or UM refers to the group of people who have one or more extra genes that produce the enzyme, so they create more enzyme than normal. The extra enzyme that UMs produce may metabolize and clear the drug from the body too rapidly and a standard dose may not have a therapeutic benefit. Or, the extra enzyme may convert the drug to its active form too rapidly and a standard dose may build up to toxic levels.

“Intermittent claudication” means the pain associated with peripheral artery disease. “Peripheral artery disease” or PAD is a type of occlusive peripheral vascular disease (PVD). PAD affects the arteries outside the heart and brain. The most common symptom of PAD is a painful cramping in the hips, thighs, or calves when walking, climbing stairs, or exercising. The pain is called intermittent claudication. When listing the symptom intermittent claudication, it is intended to include both PAD and PVD.

“Adverse event(s)” refers to any unexpected or dangerous reaction to a drug.

“Optional” and “optionally” mean that the subsequently described event or circumstance may or may not occur, and that the description includes instances where the event or circumstance occurs and instances in which it does not. For example, “optional pharmaceutical excipients” indicates that a formulation so described may or may not include pharmaceutical excipients other than those specifically stated to be present, and that the formulation so described includes instances in which the optional excipients are present and instances in which they are not.

“Treating” and “treatment” refer to any treatment of a disease in a patient and include:
1. preventing the disease from occurring in a subject which may be predisposed to the disease but has not yet been diagnosed as having it;
2. inhibiting the disease, i.e., arresting its further development;
3. inhibiting the symptoms of the disease; relieving the disease, i.e., causing regression of the disease, or relieving the symptoms of the disease.

The “patient” is a mammal, preferably a human.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Immediate release” (“IR”) refers to formulations or dosage units that rapidly dissolve in vitro and are intended to be completely dissolved and absorbed in the stomach or upper gastrointestinal tract. Conventionally, such formulations release at least 90% of the active ingredient within 30 minutes of administration.

“Sustained release” (“SR”) refers to formulations or dosage units used herein that are slowly and continuously dissolved and absorbed in the stomach and gastrointestinal tract over a period of about six hours or more. Preferred sustained release formulations are those exhibiting plasma concentrations of ranolazine suitable for no more than twice daily administration with two or less tablets per dosing as described below.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R-S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“The term “Ranolazine or the R- or S-enantiomer of ranolazine” refers to the free base of any of these 3 compounds or an ester or salt of any of these 3 compounds. The ester or salt of any of these 3 compounds can be, but is not limited to, those esters or salts named in U.S. Pat. No. 4,567,264, the specification of which is incorporated herein by reference.

The term “insulin” refers to any type of insulin from any species, including bovine, ovine, porcine, equine, and preferably human, and from any source, whether natural, synthetic, or recombinant. The term “endogenous insulin” in a patient refers to insulin produced by islet cells in the pancreas of that patient.

The term “insulin-responsive” subject refers to:
a) a subject suffering from insufficient levels of insulin, wherein the subject is capable of positively responding to enhanced levels of insulin, as evident by decreased blood glucose level of the subject after the insulin level is enhanced or
b) a subject suffering from failure to respond normally to insulin (insulin-resistance), wherein the subject positively responds to enhanced levels of insulin, as evident by decreased blood glucose level of the subject after the insulin level is enhanced.

The term “insulin secretion-deficient” subject refers to a subject capable of producing insulin in the pancreatic islet cells but in which islet cells have impaired release or production of insulin.

The term “maintaining effectiveness” of an existing anti-diabetic therapy refers to reducing the need to increase or intensify the dose of the existing anti-diabetic agent. It also refers to reducing therapy failure with the existing anti-diabetic agent, thereby reducing the need to either change the existing anti-diabetic agent or add-on another anti-diabetic agent.

The term “insulin secretion-enhancing” amount refers to the amount that is sufficient to enhance the level of endogenous insulin secreted by pancreatic islet cells.

The term “anti-diabetic” agent refers to an agent that prevents or alleviates the symptoms of diabetes.

The term “anti-diabetic therapy” refers to a course of treatment with an anti-diabetic agent, wherein the anti-diabetic agent is as defined herein.

The term “pre-diabetic” patient refers to a patient whose blood glucose levels are higher than normal but yet not high enough to be diagnosed as diabetic or a patient with impaired glucose tolerance.

The term “disposed to” diabetes mellitus refers to persons at high risk for developing diabetes mellitus. A number of risk factors are known to those of skill in the art. Some of those factors include but are not limited to, genetic factors; overweight (e.g., body mass index (BMI) greater or equal to 25 kg/m2); habitual physical inactivity, race/ethnicity (e.g., African-American, Hispanic-American, Native American, Asian-American, Pacific Islander); previously identified impaired fasting glucose or impaired glucose tolerance, hypertension (e.g., greater or equal to 140/90 mm Hg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 2002, 25(1), S5-S24.

Ranolazine is capable of forming acid and/or base salts by virtue of the presence of amino and/or carboxyl groups or groups similar thereto. The term “pharmaceutically acceptable salt” refers to salts that retain the biological effectiveness and properties of ranolazine and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amities, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Methods of the Invention

The method of the invention is based on the surprising discovery that ranolazine and its enantiomers, particularly the R-enantiomer increases insulin secretion from pancreatic islet. Insulin secretion by the pancreas is regulated by a variety of factors, among which the most important is glucose.

Glucose metabolism generates diverse coupling factors that modulate the activity of channels involved in the regulation of electrical activity and thus insulin secretion. The closure of K_{ATP} channels, resulting from an increase in the ATP-ADP ratio, leads to membrane depolarization and consequently the activation of voltage dependent calcium channels resulting in the generation of transmembrane action potentials. The depolarization leads to an increase in intercellular calcium, which plays a central role in insulin secretion. Therefore, changes in electrical activity are a necessary intermediate step in GSIS (glucose-stimulated insulin secretion).

Without being bound to the theory of the present invention, any of the following five hypotheses could contribute to the effect of ranolazine or the R-enantiomer of ranolazine to increase OSIS:
a) Ranolazine or the R-enantiomer of ranolazine by inhibiting I_{KATP} and depolarizing the β-cell and thereby activating calcium channels, increases intracellular Ca⁺⁺ followed by an increase in insulin release.
b) Ranolazine or the R-enantiomer of ranolazine by inhibiting one or any of the voltage gated K⁺ channels (K_{VX} where the X stands for 2.1 or 2.2 or 3. various isoforms of K channels) and depolarizing the β-cells and thereby activating calcium channels, increases intracellular calcium concentration followed by an increase in insulin release.
c) Ranolazine or the R-enantiomer of ranolazine by inhibiting Kᵥₓ channels to prolong the action potentials generated by β-cells due to glucose-induced inhibition of I_{KATP} to increase release of intracellular calcium and hence increased insulin secretion.
d) Ranolazine or the R-enantiomer of ranolazine by inhibiting I_{Na} and increasing the concentration of intracellular ATP by lowering the consumption of ATP, thereby inhibiting I_{KATP} causing depolarization of the β-cell followed by activating calcium channels, increases intracellular calcium concentration followed by an increase in insulin release.
e) Ranolazine or the R-enantiomer of ranolazine by inhibiting I_{Na} (peak or late) to reduce [Na⁺]_{I} (intracellular sodium) could indirectly reduce Ca⁺⁺ overload.
f.) Ranolazine or the R-enantiomer of ranolazine by reduction of pancreatic beta cell dysfunction, including Na⁺ and Ca2+ overload and induction of beta cell apoptosis and increased cell death may increase insulin secretion during chronic treatment
g.) Ranolazine or the S- or the R-enantiomer of ranolazine by the alpha-adrenergic blocking effect may contribute to the increase of insulin secretion.

In one embodiment, the present invention provides a method for enhancing endogenous insulin levels in a patient in need thereof, the method comprising administering to the patient an insulin secretion-enhancing amount of ranolazine or the R- or S-enantiomer of ranolazine.

In another embodiment, the present invention provides a method for enhancing endogenous insulin levels in a patient in need thereof which method comprises administering to the patient an insulin secretion-enhancing amount of the R-enantiomer of ranolazine.

In yet another embodiment, the present invention provides a method for reducing the amount and/or frequency of insulin administration to an insulin-responsive and insulin secretion-deficient patient, which method comprises administering to the patient an insulin secretion-enhancing amount of ranolazine or the R-enantiomer of ranolazine.

In each of the above embodiments, the patient is preferably insulin-responsive and insulin secretion-deficient. The patient may be pre-diabetic or otherwise disposed to diabetes mellitus or the patient may already suffer from type II diabetes mellitus.

### Special Properties of the R-Enantiomer

As previously mentioned the methods of the invention may be carried out with either ranolazine or the R- or S-enantiomer of ranolazine. However, various properties specific to the R-enantiomer make this compound surprisingly and uniquely suited for use. As discussed in Example 4 and as depicted in FIG. 4, the R-enantiomer is significantly more effective in increasing the secretion of insulin in response to glucose. As a consequence of this, it is anticipated that the desired therapeutic effect could be achieved using a lower dosage of the R-enantiomer than would be required with either ranolazine or the S-enantiomer of ranolazine.

Additionally, the R-enantiomer also presents pharmacokinetic advantages over ranolazine or the S-enantiomer of ranolazine. The inhibitory effects of ranolazine on CYP2D6 have been evaluated in extensive metabolizers of dextromethorphan. The study showed that ranolazine and/or metabolites partially inhibit CYP2D6. Concomitant use of ranolazine with other drugs metabolized by CYP2D6, such as tricyclic antidepressants and antipsychotics, has not been formally studied, but lower doses of the other drug than usually prescribed may be required in the presence of ranolazine. Ranolazine can inhibit the activity of CYP2D6 and thus the metabolism of drugs that are mainly metabolized by this enzyme, for example tricyclic antidepressants and some antipsychotics, may be impaired and exposure to these drugs increased. The dose of such drugs may have to be reduced when ranolazine is co-administered.

Cytochrome P450s (CYP) generally comprise the major enzymes responsible for oxidative metabolism of drugs. The CYP isozyme CYP2D6 specifically has a wide range of activity within human populations, with inter-individual rates of metabolism differing by more than 10,000 fold. Most individuals are extensive metabolizers (EM), able to metabolize CYP2D6 substrates extensively, whereas 7-10% of Caucasian individuals are poor metabolizers (PM), producing no functional CYP2D6 enzyme. Poor metabolizers across all populations, including Asians and African Americans, comprise 2-10%.

DNA polymorphisms have been identified in the genes encoding a number of CYP isozymes, leading to wide interindividual variation in drug clearance. CYP2D6 metabolizes a significant number of clinically used medications, and genetic variants of the CYP2D6 isozyme that result in varying levels of metabolic activity are of clinical importance in some settings. The exact nature of the clinical effect caused by polymorphisms of the gene depends on the drug in question and the specific variant alleles expressed, as individual variants result in differing phenotypes with a range of levels of enzymatic activity.

Inter-individual variability in drug metabolism poses a challenge in predicting dosing, safety, and efficacy of a drug. For example, pharmacokinetic factors, as well as substantial intersubject pharmacodynamic variability, have been proposed as a factor in cases of therapeutic failure of methylphenidate. [DeVane, et al., Journal of Clinical Psychopharmacology, 20(3), 347 (2000)]

Individuals lacking the expression of a polymorphic drug-metabolizing enzyme (commonly referred to as “poor metabolizers” or PMs) will have higher drug exposure if those drugs are metabolized by those polymorphic enzymes, which could lead to exaggerated pharmacology or enhanced side effects relative to the intermediate metabolizer and extensive metabolizer (EM) subjects given the same dose. Alternatively, if a polymorphic enzyme forms a particular metabolite that contributes to the activity of a drug, then different efficacy profiles might be observed in EM and PM patients. [Gibbs, et al., Drug Metabolism and Disposition 34(9), 1516-1522 (2006)]

Of the identified polymorphic enzymes involved in drug metabolism, CYP2D6 is considered one of the most important, with a substrate specificity typical of many new chemical entities. An estimated 20 to 25% of all drugs in clinical use are metabolized at least in part by CYP2D6. The frequency of CYP2D6 PMs in the populations depends on race and is reported to be approximately 1% for Asians and 5 to 10% of Caucasians. [Gibbs, et al., Drug Metabolism and Disposition 34(9), 1516-1522 (2006)] The primarily hepatic expression of this enzyme governs first pass metabolism after oral drug administration, whereas the low levels of intestinal expression do not appear to be important. Numerous studies have characterized the impact of CYP2D6 polymorphism on substrate area under the curve (AUC) in EM and PM subjects.

Known cardiovascular drug substrates of CYP2D6 include, but are not limited to:
Antiarrhythmics: amiodarone, encamide, flecamide, lidocaine, and mexiletine;
Antihypertensives: captopril, clonidine, debrisoquine, gaunoxan, indapamide, N-propylajmaline, procainamide, propafenone, and spartein;
Beta blockers: alprenolol, bisoprolol, bufuralol, bupranolol, carvedilol, labetalol, metoprolol, pindolol, propanolol, and timolol;
Calcium channel blockers: cinnarizine, flunarizine, nimodipine, nitrendipine, and perhexyline; and
Antidiabetic drugs: phenformin.

Although the poor metabolizer phenotype is known to be caused by two null alleles leading to absence of functional CYP2D6 protein, the large variability among individuals with functional alleles remains mostly unexplained.

Many drug interactions result from inhibition (e.g., by competing for the same enzyme binding site) or induction (increased enzyme protein synthesis) of CYP enzymes. Unlike other CYP isozymes, CYP2D6 is not considered to be inductible (e.g., by drugs such as riampicin). However, it is subject to inhibition. Some drugs such as paroxetine inhibit CYP2D6 so strongly that up to 80% of EMs are ‘converted’ to PMs i.e., markedly reducing ability to metabolize CYP2D6 substrates. Other strong CYP2D6 inhibitors include fluoxetine, terbinafine and thioridazine. Weaker inhibitors include tricyclic antidepressants and citalopram.

In general, PMs have higher parent drug concentrations and may develop toxic concentrations with standard doses. In some cases, when the CYP2D6 metabolite is more active than the parent, reduced drug effect may occur because of reduced production of the active metabolite. Ultra-rapid metabolizers eliminate CYP2D6 substrates very quickly and may not achieve therapeutic concentrations with standard doses.

Thus, there are strong advantages in the use of drugs which display reduced inhibition of CYP2D6. When given to patients suffering from one or more cardiovascular diseases or cardiac disease symptoms, use of these agents reduces the inter-individual variation among patients, and reduces possible adverse events in poor metabolizer patients. Reduced inhibition of CYP2D6 also may reduce the inter-individual variability seen among patients, and reduce possible adverse events in poor metabolizer patients when the patient is prescribed a combination of drugs for cardiovascular symptoms, especially when the combination of drugs are metabolized via CYP2D6.

As discussed in Examples 6 and 7, the R-enantiomer of Ranolazine has been shown to inhibit CYP2D6 to a much lesser degree than either ranolazine or the S-enantiomer of ranolazine.

Given the surprising advantages presented by the R-enantiomer, both with respect to its augmented ability to increase glucose induces insulin excretion and its diminished capacity to inhibit CYP2D6, in one aspect, this invention provides for a method for treating a patient suffering from one or more cardiovascular diseases or cardiovascular disease symptoms, which methods reduce adverse events and/or drug-drug interactions caused by possible dysfunction of the CYP2D6 enzyme, comprising administering the R-enantiomer of ranolazine to these patients.

Patients presenting themselves with one or more cardiovascular disease events or symptoms include, but are not limited to, those who are being treated for one or more of the following: angina including stable angina, unstable angina (UA), exercised-induced angina, variant angina, arrhythmias, intermittent claudication, myocardial infarction including STEW and non-STE myocardial infarction (NSTEMI), heart failure including congestive (or chronic) heart failure, acute heart failure, or recurrent ischemia.

Other conditions which are treatable using the method of the invention, include, but are not limited to, heart failure, including congestive heart failure, acute heart failure, myocardial infarction, and the like, arrhythmias including treatment of supra ventricular tachycardias such as atrial fibrillation, atrial flutter, AV nodal reentrant tachycardia, atrial tachycardia, and the ventricular tachycardias (VTs), including idiopathic ventricular tachycardia, ventricular fibrillation, pre-excitation syndrome, and Torsade de Pointes (TdP), angina, including exercise-induced angina, variant angina, stable angina, unstable angina, acute coronary syndrome, and the like, and peripheral artery disease, including intermittent claudication.

### Special Properties of the S-Enantiomer

Although the R-enantiomer is highly suited for use in the methods of the invention, is should also be noted that the S-enantiomer also possesses unique properties that make it ideal for use as an anti-ischemic and anti-arrhythmic agent. As discussed in Example 8, 9, and 10, the S-enantiomer of ranolazine is a more potent inhibitor of the late sodium channel, I_{Kr}, β₁-AR, and β₂-AR than either the R-enantiomer or racemic ranolazine. Consequently, the S-enantiomer is potentially a stronger and better antianginal and anti-arrhythmic drug than either the R-enantiomer or racemic ranolazine.

Given the differing properties of the two enantiomers, it is another aspect of the invention wherein a dosage form is provided which comprises both the R— and the S-enantiomer of ranolazine in non-equal amounts. The advantage of such a non-racemic combination is that by varying the amount of either the S- or R-enantiomers one of skill in the art could prepare a formulation that provided better efficacy for treating insulin deficient patients, or patients suffering from cardiovascular disease, or patients suffering from both.

Based on the surprising advantages presented by the S-enantiomer with respect to its increase potency for cardiovascular indications in yet another aspect, this invention provides for a method for treating a patient suffering from one or more cardiovascular diseases or cardiovascular disease symptoms, which methods reduce adverse events, comprising administering a therapeutically effective minimum dose of the S-enantiomer of ranolazine to these patients.

### Co-Administration

According to this invention, ranolazine or the R- or S-enantiomer of ranolazine can be used in combination with another anti-diabetic agent, or in combination with more than one anti-diabetic agent, to treat diabetes mellitus and/or to improve glycemic control in patients in need of treatment. The compounds can be administered individually, or can be combined in a single formulation, for example as a tablet, capsule, syrup, solution, as well as controlled release formulations. The dosage of each agent will vary depending upon the severity of the disease, the frequency of administration, the particular agents and combinations utilized, and other factors routinely considered by an attending medical practitioner.

Thus in another embodiment, the present invention provides a method for treating an insulin-responsive and insulin secretion-deficient patient, which method comprises administering to the patient an insulin secretion-enhancing amount of ranolazine or the R-enantiomer of ranolazine in combination with at least one anti-diabetic agent.

In another embodiment, the present invention provides a method for maintaining effectiveness of anti-diabetic therapy in a patient, wherein the method comprises administering to the patient an insulin secretion-enhancing amount of ranolazine or the R-enantiomer of ranolazine in combination with the anti-diabetic.

In another embodiment, the present invention provides a composition comprising an insulin secretion-enhancing amount of ranolazine or the R-enantiomer of ranolazine and at least one anti-diabetic agent.

In another embodiment, the present invention also provides a method comprising co-administering an insulin secretion-enhancing amount of ranolazine or the R-enantiomer of ranolazine and at least one anti-diabetic agent. Co-administration can be in the form of a single formulation (combining, for example, ranolazine or the R-enantiomer of ranolazine and another anti-diabetic agent with pharmaceutically acceptable excipients, optionally segregating the two active ingredients in different excipient mixtures designed to independently control their respective release rates and durations) or by independent administration of separate formulations containing the active agents.

“Co-administration” further includes concurrent administration (administration of ranolazine or the R-enantiomer of ranolazine and other anti-diabetic agent at the same time) and time varied administration (administration of ranolazine or the R-enantiomer of ranolazine at a time different from that of the other anti-diabetic agent), as long as both the ranolazine or the R-enantiomer of ranolazine and other anti-diabetic agent are present in the serum in therapeutically effective concentrations during at least partially overlapping times.

Accordingly, in some aspects, ranolazine or the R-enantiomer of ranolazine and the anti-diabetic agent(s) can be administered in a single formulation. In some other aspects, ranolazine or the R-enantiomer of ranolazine and the anti-diabetic agent(s) can be administered individually but simultaneously. In some other aspects, ranolazine or the R-enantiomer of ranolazine and the anti-diabetic agent(s) can be administered individually but sequentially.

In some aspects, said anti-diabetic agent is selected from the group consisting of sulfonylureas, DPP-IV inhibitors, biguanides, thiazolidindiones, alpha-glucosidase inhibitors, incretin mimetics, PPAR gamma modulators, dual PPARalpha/gamma agonists, RXR modulators, SGLT2 inhibitors, aP2 inhibitors, insulin sensitizers, PTP-IB inhibitors, GSK-3 inhibitors, DP4 inhibitors, insulin sensitizers, insulin, meglitinide, PTP1B inhibitors, glycogen phosphorylase inhibitors, glucose-6-phosphatase inhibitor, and amylin analogs.

Examples of sulfonylureas include but are not limited to tolbutamide, tolazamide, acetohexamide, chlorpropamide, glyburide, glipizide, glimepiride, gliclazide, gliquidone, etc. Examples of biguanides include but are not limited to metformin, phenformin, etc. Examples of meglitinides include but are not limited to repaglinide, nateglinide, etc. Examples of PPAR gamma modulators include but are not limited to thiazolidinediones such as rosiglitazone, pioglitazone, troglitazone, etc. Examples of alpha-glucosidase inhibitors include but are not limited to miglitol, acarbose, etc. Examples of incretin mimetics include but are not limited to exenatide. Examples of DPP-IV inhibitors include but are not limited to vildagliptin, sitagliptin, etc. Examples of amylin analogs include but are not limited to pramlintide.

In some preferred aspects, the present invention provides a composition comprising an insulin secretion-enhancing amount of ranolazine or the R-enantiomer of ranolazine and at least one anti-diabetic agent, wherein said agent is selected from the group consisting of metformin, phenformin, buformin, chlorpropamide, glisoxepid, glyburide, acetohexamide, chlorpropamide, glibornuride, tolbutamide, tolazamide, glipizide, glimepiride, gliclazide, gliquidone, glyhexamide, phenbentamide, tolcyclamide, troglitazone, pioglitazone, rosiglitazone, miglitol, acarbose, exenatide, vildagliptin, sitagliptin, repaglinide, pramlintide, and nateglinide.

### Utility Testing and Administration

### General Utility

The method of the invention is useful for increasing glucose stimulated insulin secretion in patients that are insulin-responsive and insulin secretion-deficient. The method is also useful with respect to the R-enantiomer of ranolazine which is effective for treating mammals for various disease states, such as for example, heart failure, including congestive heart failure, acute heart failure, ischemia, recurrent ischemia, myocardial infarction, STEMS and NSTEMI, and the like, arrhythmias, angina, including exercise-induced angina, variant angina, stable angina, unstable angina, acute coronary syndrome, NSTEACS, and the like, diabetes, and intermittent claudication

### Pharmaceutical Compositions and Administration

Ranolazine or the R- or S-enantiomer of ranolazine is usually administered in the form of a pharmaceutical composition. This invention therefore provides pharmaceutical compositions that contain, as the active ingredient, ranolazine, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. Ranolazine may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

The ranolazine or the R- or S-enantiomer of ranolazine may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

### Suppositories, Suspensions, Sub-Q, and Topical

Representative examples of suppositories, suspensions, subcutaneous formulations, and topical preparations are as below.

Suppositories, each containing 25 mg of active ingredient can be made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions, each containing 50 mg of active ingredient per 5.0 mL dose can be made as follows:

| Ingredient | Amount | |
|---|---|---|
| Active Ingredient | 50 | mg |
| Xanthan gum | 4.0 | mg |
| Sodium carboxymethyl cellulose | 11% | |
| Microcrystalline cellulose | 50 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with sonic of the water and added with stirring. Sufficient water is then added to produce the required volume.

A subcutaneous formulation can be prepared as follows:

| Ingredient | Amount | |
|---|---|---|
| Active Ingredient | 5.0 | mg |
| Corn oil | 1.0 | mL |

A topical preparation having the following composition can be prepared:

| Ingredient | Amount (Grams) |
|---|---|
| Active Ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### Oral Formulations

Oral administration is the preferred route for administration of ranolazine and the R- or S-enantiomer of ranolazine. Administration may be via capsule or enteric coated tablets, or the like. In making the pharmaceutical compositions that include ranolazine, or the R- or S-enantiomer of ranolazine, the active ingredient is usually diluted by an excipient and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 50% by weight of the active compound, soft and hard gelatin capsules, sterile injectable solutions, and sterile packaged powders.

Representative examples of capsules and tablets are as below.

Hard gelatin capsules containing the following ingredients can be prepared:

| Ingredient | Quantity (mg/capsule) |
|---|---|
| Active Ingredient | 30.0 |
| Starch | 305.0 |
| Magnesium stearate | 5.0 |

The above ingredients are mixed and filled into hard gelatin capsules.

A tablet formula can be prepared using the ingredients below:

| Ingredient | Quantity (mg/tablet) |
|---|---|
| Active Ingredient | 30.0 |
| Cellulose, microcrystalline | 305.0 |
| Colloidal silicon dioxide | 5.0 |
| Stearic acid | 5.0 |

The components are blended and compressed to form tablets.

Tablets, each containing 30 mg of active ingredient, can be prepared as follows:

| Ingredient | Quantity (mg/tablet) |
|---|---|
| Active Ingredient | 30.0 mg |
| Starch | 45.0 mg |
| Microcrystalline cellulose | 35.0 mg |
| Polyvinylpyrrolidone (as 10% solution in sterile | 4.0 mg |
| water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

### Formulation for Inhalation or Insufflation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

A representative example of dry powder inhaler formulation can be prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Parental Administration

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the invention in the required amount in the appropriate solvent with various other ingredients as enumerated above, as required, followed by filtration and sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

A representative example of an injectable preparation having the following composition can be prepared:

| Ingredients | Amount |
|---|---|
| Active ingredient | 2.0 mg/mL |
| Mannitol, USP | 50 mg/mL |
| Gluconic acid, USP | q.s. (pH 5-6) |
| water (distilled, sterile) | q.s. to 1.0 mL |
| Nitrogen Gas, NF | q.s. |

### IV Administration and Stent

The intravenous formulation of ranolazine or the R- or S-enantiomer of ranolazine is manufactured via an aseptic fill process as follows. In a suitable vessel, the required amount of Dextrose Monohydrate is dissolved in Water for Injection (WFI) at approximately 78% of the final batch weight. With continuous stirring, the required amount of ranolazine free base is added to the dextrose solution. To facilitate the dissolution of ranolazine, the solution pH is adjusted to a target of 3.88-3.92 with 0.1N or 1N Hydrochloric Acid solution. Additionally, 0.1N HCl or 1.0N NaOH may be utilized to make the final adjustment of solution to the target pH of 3.88-3.92. After ranolazine is dissolved, the batch is adjusted to the final weight with WFI. Upon confirmation that the in-process specifications have been met, the ranolazine or the R- or S-enantiomer of ranolazine bulk solution is sterilized by sterile filtration through two 0.2 μm sterile filters. Subsequently, the sterile ranolazine or the R- or S-enantiomer of ranolazine bulk solution is aseptically filled into sterile glass vials and aseptically stoppered with sterile stoppers. The stoppered vials are then sealed with clean flip-top aluminum seals.

Ranolazine or the R- or S-enantiomer of ranolazine may be impregnated into a stent by diffusion, for example, or coated onto the stent such as in a gel form, for example, using procedures known to one of skill in the art in light of the present disclosure.

### Unit Dosage Forms

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule).

Ranolazine or the R- or S-enantiomer of ranolazine is effective over a wide dosage range and are generally administered in a pharmaceutically effective amount. Preferably, for oral administration, each dosage unit contains from 10 mg to 3 g of ranolazine or the R- or S-enantiomer of ranolazine, more preferably 10 mg to 2 g of ranolazine or the R- or S-enantiomer of ranolazine, more preferably 10 mg to 1500 mg, more preferably from 10 mg to 1000 mg, more preferably from 10 mg to 700 mg, and for parenteral administration, preferably from 10 mg to 700 mg, more preferably about 50 mg to 200 mg.

It will be understood, however, that the amount of ranolazine or the R- or S-enantiomer of ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like. A unit dosage form typically will be administered once, twice, three times, or four times daily. The unit dosage form may be taken prior to, with, or after meals.

### Quick and Sustained Release

In one embodiment, the ranolazine or the R- or S-enantiomer of ranolazine is formulated so as to provide quick, sustained or delayed release of the active ingredient after administration to the patient, especially sustained release formulations by employing procedures known in the art. Unless otherwise stated, the ranolazine plasma concentrations used in the specification and examples refer to ranolazine or the R- or S-enantiomer of ranolazine free base. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

It is contemplated that ranolazine or the R- or S-enantiomer of ranolazine is formulated so as to provide a combination of quick and sustained release or delayed release. In this embodiment, a sustained or controlled release core may be covered with an immediate release layer. This type of formulation would be advantageous as it could be taken prior to a large meal thereby providing increased insulin secretion in response to the meal immediately following administration with sustained release thereafter. This type of coating and core formulation may also be used to prepare formulations that include one or more anti-diabetic agents as discussed above.

Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The preferred sustained release formulations of this invention are preferably in the form of a compressed tablet comprising an intimate mixture of compound and a partially neutralized pH-dependent binder that controls the rate of dissolution in aqueous media across the range of pH in the stomach (typically approximately 2) and in the intestine (typically approximately about 5.5). An example of a sustained release formulation is disclosed in U.S. Pat. Nos. 6,303,607; 6,479,496; 6,369,062; and 6,525,057, the complete disclosures of which are hereby incorporated by reference.

To provide for a sustained release of ranolazine or the R- or S-enantiomer of ranolazine, one or more pH-dependent binders are chosen to control the dissolution profile of the compound so that the formulation releases the drug slowly and continuously as the formulation passed through the stomach and gastrointestinal tract. The dissolution control capacity of the pH-dependent binder(s) is particularly important in a sustained release formulation because a sustained release formulation that contains sufficient compound for twice daily administration may cause untoward side effects if the compound is released too rapidly (“dose-dumping”).

Accordingly, the pH-dependent binders suitable for use in this invention are those which inhibit rapid release of drug from a tablet during its residence in the stomach (where the pH is below about 4.5), and which promotes the release of a therapeutic amount of compound from the dosage form in the lower gastrointestinal tract (where the pH is generally greater than about 4.5). Many materials known in the pharmaceutical art as “enteric” binders and coating agents have the desired pH dissolution properties. These include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof.

Preferred pH-dependent binder materials that can be used in conjunction with the compound to create a sustained release formulation are methacrylic acid copolymers. Methacrylic acid copolymers are copolymers of methacrylic acid with neutral acrylate or methacrylate esters such as ethyl acrylate or methyl methacrylate. A most preferred copolymer is methacrylic acid copolymer, Type C, USP (which is a copolymer of methacrylic acid and ethyl acrylate having between 46.0% and 50.6% methacrylic acid units). Such a copolymer is commercially available, from Röhm Pharma as Eudragit® L 100-55 (as a powder) or L30D-55 (as a 30% dispersion in water). Other pH-dependent binder materials which may be used alone or in combination in a sustained release formulation dosage form include hydroxypropyl cellulose phthalate, hydroxypropyl methylcellulose phthalate, cellulose acetate phthalate, polyvinylacetate phthalate, polyvinylpyrrolidone phthalate, and the like.

One or more pH-independent binders may be in used in sustained release formulations in oral dosage forms. It is to be noted that pH-dependent binders and viscosity enhancing agents such as hydroxypropyl methylcellulose, hydroxypropyl cellulose, methylcellulose, polyvinylpyrrolidone, neutral poly(meth)acrylate esters, and the like, may not themselves provide the required dissolution control provided by the identified pH-dependent binders. The pH-independent binders may be present in the formulation of this invention in an amount ranging from about 1 to about 10 wt %, and preferably in amount ranging from about 1 to about 3 wt % and most preferably about 2.0 wt %.

As shown in Table 1, ranolazine or the R- or S-enantiomer of ranolazine is relatively insoluble in aqueous solutions having a pH above about 6.5, while the solubility begins to increase dramatically below about pH 6.

TABLE 1

| Solution pH | Solubility (mg/mL) | USP Solubility Class |
|---|---|---|
| 4.81 | 161 | Freely Soluble |
| 4.89 | 73.8 | Soluble |
| 4.90 | 76.4 | Soluble |
| 5.04 | 49.4 | Soluble |
| 5.35 | 16.7 | Sparingly Soluble |
| 5.82 | 5.48 | Slightly soluble |
| 6.46 | 1.63 | Slightly soluble |
| 6.73 | 0.83 | Very slightly soluble |
| 7.08 | 0.39 | Very slightly soluble |
| 7.59 | 0.24 | Very slightly soluble |
| (unbuffered water) | | |
| 7.79 | 0.17 | Very slightly soluble |
| 12.66 | 0.18 | Very slightly soluble |

Increasing the pH-dependent binder content in the formulation decreases the release rate of the sustained release form of the compound from the formulation at pH is below 4.5 typical of the pH found in the stomach. The enteric coating formed by the binder is less soluble and increases the relative release rate above pH 4.5, where the solubility of compound is lower. A proper selection of the pH-dependent binder allows for a quicker release rate of the compound from the formulation above pH 4.5, while greatly affecting the release rate at low pH. Partial neutralization of the binder facilitates the conversion of the binder into a latex like film which forms around the individual granules. Accordingly, the type and the quantity of the pH-dependent binder and amount of the partial neutralization composition are chosen to closely control the rate of dissolution of compound from the formulation.

The dosage forms of this invention should have a quantity of pH-dependent binders sufficient to produce a sustained release formulation from which the release rate of the compound is controlled such that at low pHs (below about 4.5) the rate of dissolution is significantly slowed. In the case of methacrylic acid copolymer, type C, USP (Eudragit® L 100-55), a suitable quantity of pH-dependent binder is between 5% and 15%. The pH dependent binder will typically have from about 1 to about 20% of the binder methacrylic acid carboxyl groups neutralized. However, it is preferred that the degree of neutralization ranges from about 3 to 6%. The sustained release formulation may also contain pharmaceutical excipients intimately admixed with the compound and the pH-dependent binder.

Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters (e.g. the methyl methacrylate/ethyl acrylate copolymers sold under the trademark Eudragit® NE by Röhm Pharma, starch, gelatin, sugars carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as tale and magnesium stearate) and other tableting aids are also optionally present.

The sustained release formulations of this invention have an active compound content of about 35% by weight to about 95% or more by weight, about 50% by weight to about 95% or more by weight, more preferably between about 70% to about 90% by weight and most preferably from about 70 to about 80% by weight; a pH-dependent binder content of between 5% and 40%, preferably between 5% and 25%, and more preferably between 5% and 15%; with the remainder of the dosage form comprising pH-independent binders, fillers, and other optional excipients.

One particularly preferred sustained release formulations of this invention is shown below in Table 2.

TABLE 2

| | Weight | Preferred | Most |
|---|---|---|---|
| Ingredient | Range (%) | Range (%) | Preferred |
| Active ingredient | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release formulations of this invention are prepared as follows: compound and pH-dependent binder and any optional excipients are intimately mixed (dry-blended). The dry-blended mixture is then granulated in the presence of an aqueous solution of a strong base that is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, preferably sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl, methylcellulose, cationic methacrylate copolymers (dimethylaminoethyl methacrylate/methyl-butyl methacrylate copolymers—Eudragit® E—Röhm. Pharma), and the like. These film-forming agents may optionally contain colorants, plasticizers, and other supplemental ingredients.

The compressed tablets preferably have a hardness sufficient to withstand 8 Kp compression. The tablet size will depend primarily upon the amount of compound in the tablet. The tablets will include from 300 to 1100 mg of compound free base. Preferably, the tablets will include amounts of compound free base ranging from 400-600 mg, 650-850 mg, and 900-1100 mg.

In order to influence the dissolution rate, the time during which the compound containing powder is wet mixed is controlled. Preferably the total powder mix time, i.e. the time during which the powder is exposed to sodium hydroxide solution, will range from 1 to 10 minutes and preferably from 2 to 5 minutes. Following granulation, the particles are removed from the granulator and placed in a fluid bed dryer for drying at about 60° C.

It has been found that these methods produce sustained release formulations that provide lower peak plasma levels and yet effective plasma concentrations of compound for up to 12 hours and more after administration, when the compound is used as its free base, rather than as the more pharmaceutically common dihydrochloride salt or as another salt or ester. The use of free base affords at least one advantage: The proportion of compound in the tablet can be increased, since the molecular weight of the free base is only 85% that of the dihydrochloride. In this manner, delivery of an effective amount of compound is achieved while limiting the physical size of the dosage unit.

### Unit Dosage Forms Specific to the R-Enantiomer

Intravenous Formulation

In one aspect, the invention provides an intravenous (IV) solution comprising a selected concentration of R-ranolazine. Specifically, the IV solution preferably comprises about 1.5 to about 3.0 mg of R-ranolazine per milliliter of a pharmaceutically acceptable aqueous solution, more preferably about 1.8 to about 2.2 mg and even more preferably about 2 mg.

Oral Formulation

In one embodiment, a formulation of R-ranolazine is an oral formulation. In one embodiment, an oral formulation of R-ranolazine is a tablet. In one embodiment, the tablet of R-ranolazine is up to 500 mg. In a preferred embodiment, the R-ranolazine tablet is 375 mg, and/or 500 mg.

The oral formulation of ranolazine is thoroughly discussed in U.S. Pat. No. 6,303,607 and U.S. Publication No. 2003/0220344, which are both incorporated herein by reference in their entirety.

The oral sustained release R-ranolazine dosage formulations of this invention are administered one, twice, or three times in a 24 hour period in order to maintain a plasma ranolazine level above the threshold therapeutic level and below the maximally tolerated levels, which is preferably a plasma level of about 550 to 7500 ng base/mL in a patient.

In a preferred embodiment, the plasma level of ranolazine ranges about 1500-3500 ng base/mL.

In order to achieve the preferred plasma ranolazine level, it is preferred that the oral R-ranolazine dosage forms described herein are administered once or twice daily. If the dosage forms are administered twice daily, then it is preferred that the oral R-ranolazine dosage forms are administered at about twelve hour intervals.

In addition to formulating and administering oral sustained release dosage forms of this invention in a manner that controls the plasma ranolazine levels, it is also important to minimize the difference between peak and trough plasma ranolazine levels. The peak plasma ranolazine levels are typically achieved at from about 30 minutes to eight hours or more after initially ingesting the dosage form while trough plasma ranolazine levels are achieved at about the time of ingestion of the next scheduled dosage form. It is preferred that the sustained release dosage forms of this invention are administered in a manner that allows for a peak ranolazine level no more than 8 times greater than the trough ranolazine level, preferably no more than 4 times greater than the trough ranolazine level, preferably no more than 3 times greater than the trough ranolazine level, and most preferably no greater than 2 times trough ranolazine level.

The sustained release R-ranolazine formulations of this invention provide the therapeutic advantage of minimizing variations in ranolazine plasma concentration while permitting, at most, twice-daily administration. The formulation may be administered alone, or (at least initially) in combination with an immediate release formulation if rapid achievement of a therapeutically effective plasma concentration of ranolazine is desired, or by soluble IV formulations and oral dosage forms.

The invention is further understood by reference to the following examples, which are intended to be purely exemplary of the invention. The present invention is not limited in scope by the exemplified aspects, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Effect of Ranolazine or the R- or S-enantiomers of Ranolazine on GSIS in rat Isolated Pancreatic Islets

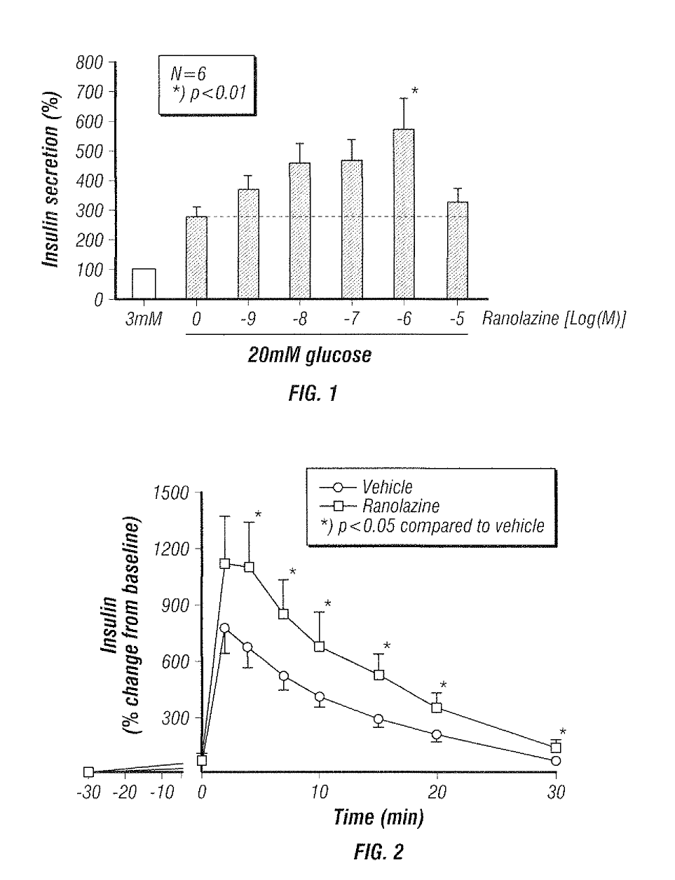

FIG. 1 shows the effect of ranolazine on GSIS in rat isolated pancreatic islets. Rat islets were isolated from male Sprague-Dawley (SD) rats (8-12 weeks old, n=6) as described by Yang Z. et al. Transplantation 2004, 77, 55-60 and maintained in complete RPMI1640 overnight. Insulin secretion assay was performed essentially as previously described by Liu D. et al. Steroids 2006, 71, 691-699. Briefly, before the experiment, the islets were pre-incubated in Krebs-Ringer bicarbonate buffer (KRB; 1.29 mM NaCl, 4.8 mM KCl, 1.2 mM MgSO₄, 1.2 mM KH₂PO₄, 2.5 mM CaCl₂, 5 mM NaHCO₃, 0.1% BSA, 10 mM HEPES, pH 7.4) containing 3 mM glucose for 30 min, after which islets were then washed and incubated in triplicate in 24-well plate (50 islets/well), in oxygenated KRB buffer with 3 mM glucose or 20 mM glucose in the presence of various concentrations of ranolazine or vehicle for 60 min at 37° C. Insulin secreted in experimental samples was measured by a RIA kit (Mercodia, N.C.).

All insulin secretion data in the present study were normalized to insulin secretion per islet. There was a biphasic response for insulin secretion to ranolazine. At lower concentrations (1 nM to 1 μM) ranolazine increased (2-5 fold) insulin secretion in a concentration-dependent manner. At a concentration of 10 μM ranolazine had no significant effect on GSIS, that is, the amount of insulin release was not very different from that observed with 20 mM glucose.

Using the procedure set forth above, the R- and S-enantiomers of ranolazine were tested in isolated rat pancreatic islets to show the effect of the R- and S-enantiomers of ranolazine on GSIS.

### Example 2

### Effect of Ranolazine on Insulin Levels in Rats

FIG. 2 shows plasma insulin levels during an intravenous glucose tolerance test (IVGTT) performed in normal SD rats. Each rat was subjected to an IVGTT according to the method of Hendrick et al. Metabolism 42, (1):1-6, 1993. The rats were fasted overnight before administration of the test. One group of eleven rats was given only saline prior to glucose load while the second group of seven rats was given ranolazine prior to glucose load. Glucose was administered at time zero while ranolazine was administered 30 minutes prior to glucose at a dose of 15 mg/kg of body weight. Then, the insulin concentrations in the blood were measured at −30, 0, 2, 4, 7, 15 and 30 minutes. As seen from the plot of time versus insulin levels relative to baseline, insulin levels were higher in rats treated with ranolazine as compared to vehicle treated rats.

### Example 3

### Effect of Ranolazine Enantiomers on Insulin Levels in Rats

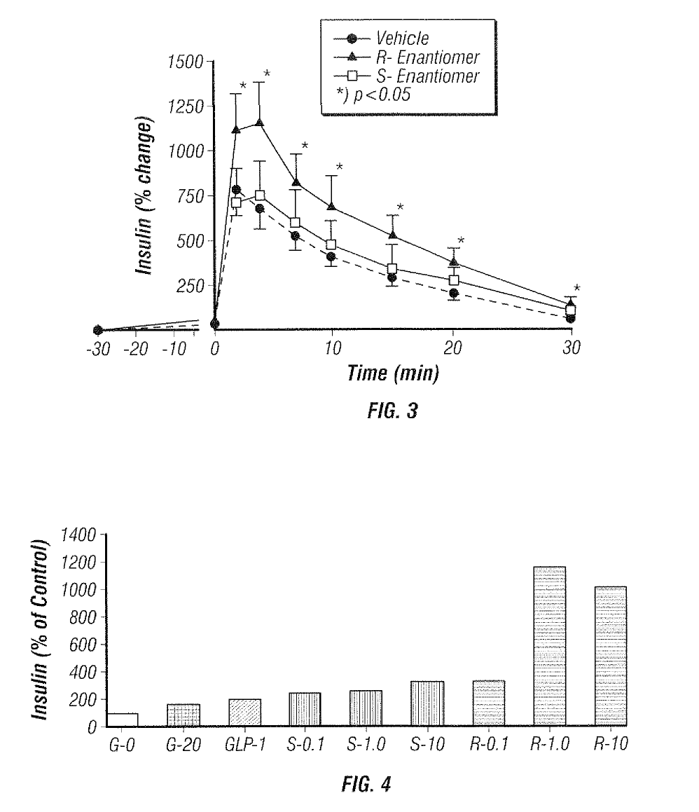

FIG. 3 shows insulin levels during an intravenous glucose tolerance test (IVGTT) performed in normal SD rats. The procedure used was that described in Example 2 above. As seen from the plot of time versus insulin levels relative to baseline, insulin levels were higher in rats treated with the R-enantiomer of ranolazine at 15 mg/kg as compared to vehicle treated rats. The insulin response for the S-enantiomer was not different from the vehicle treated rats.

### Example 4

### Effect of the R- or S-Enantiomer of Ranolazine on GSIS in Human Islets

FIG. 4 shows the effect of the R-enantiomer of ranolazine (designated “R-”) and the S-enantiomer of ranolazine (designated “S-”) on GSIS in human isolated pancreatic islets from one donor. The procedure used for the human islets was the same as the procedure used in Example 1 above, except that the incubation period was 30 minutes, instead of the 60 minutes used in Example 1.

### Example 5

### Effect of Ranolazine on GSIS in Human Islets

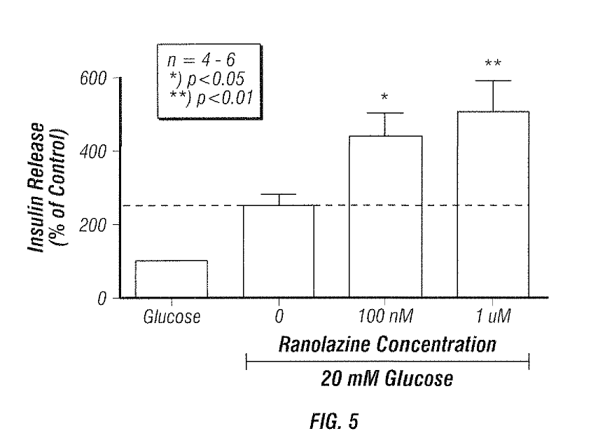

FIG. 5 shows the effect of (±) ranolazine on GSIS in isolated human pancreatic islets. The procedure used for the human islets was the same as the procedure used in Example 1 above, except that the incubation period was 30 minutes, instead of the 60 minutes used in Example 1. “n” is the number of donors.

### Example 6

### Effect of Racemic Ranolazine on CYP2D6

The effects of Racemic ranolazine on CYP2D6 was tested using an incubation mixture containing potassium phosphate, pH 7.4 (final concentration=100 mM), a NADPH (nicotinamide adenine dinucleotide phosphate, reduced) regenerating mixture containing 0.163 mM NADP (nicotinamide adenine dinucleotide phosphate), 1.64 mM G-6-P (glucose-6-phosphate), and 0.4 units/mL glucose-6-phosphate dehydrogenase, CYP2D6-specific marker substrates (bufuralol, 12.5 μM, or dextromethorphan, 10 μM), ranolazine (10, 25, 50, 100, 250, 500, 1000 μM), or the known CYP2D6 inhibitor (quinidine, 1 μM), and human liver microsomal (HUM) proteins (0.5 mg/mL). The substrate, buffer and enzyme were pre-warmed at 37° C. The pre-warmed NADPH regenerating system and were then added and the samples gently mixed and incubated for 30 minutes. The effect of ranolazine on the CYP2D6 marker reactions to form 1-hydroxy-bufurolol, and dextrophan was monitored by tandem mass spectrometric assays. A known CYP2D6 inhibitor, quinidine, was included as a positive control. The negative control samples consisted of all components except ranolazine or known inhibitors. Incubations were carried out in duplicate. Racemic ranolazine (in free base form) was found to have an IC₅₀ (μM) of 324.

### Example 7

### Effect of the R- and S-Enantiomers of Ranolazine on CYP2D6

The incubation mixture contained potassium phosphate, pH 7.2 (final concentration=100 mM), magnesium chloride (5 mM), NADPH (1 mM), CYP2D6-specific marker substrate (bufuralol, 25 μM), (R) or (S)-ranolazine (10, 25, 100, 250, 1000 μM), or known CYP2D6 inhibitor (quinidine, 5 μM), and human liver microsomal (HLM) proteins. AU reagents except NADPH were combined and preincubated at 37° C. for 2-3 min. Reactions were initiated by addition of NADPH and incubated 20 minutes. The CYP2D6 marker reaction, bufuralol 1-hydroxylation, was monitored. A known CYP2D6 inhibitor, quinidine, was included as a positive control. The negative control samples consisted of all components but not ranolazine. Incubations were run in triplicate for each condition except for positive controls, which were carried out in duplicate. R-ranolazine (in free base form) was found to have an IC₅₀ (μM) of 430. S-ranolazine (in free base form) was found to have an IC₅₀ (μM) of 130.

### Example 8

### Effect of Ranolazine and the R- and S-Enantiomers of Ranolazine β1 and β2-Adrenergic Receptors

The objective of this example is to present the affinities and potencies of ranolazine and its S- and R-enantiomers for β₁ and β₂-adrenergic receptors (ARs) in various tissues and cell lines. Using radioligand binding techniques, competition of the compounds for specific radioligand binding to β₁- and β₂-ARs was determined using membranes prepared from rat ventricle and guinea-pig lung, respectively. The affinities (Kᵢ values) of ranolazine were 8.6 and 14.8 μM of β₁- and β₂-ARs, respectively. The S-enantiomer has higher affinities with K values of 4.8 and 6.7 μM whereas the R-enantiomer has much lower affinities with K values of >100 and 39.0 μM for β₁- and β₂-ARs, respectively.

The effect of ranolazine and its S- and R-enantiomers on cAMP accumulations in rat C6 glioma cells (expressing mostly β₁-ARS) and DDT₁ MF-2 hamster smooth muscles cells (expressing mostly β₂-ARs) were determined. None of the compounds showed any agonist activity, i.e., they did not increase cAMP accumulation by themselves. However, in the presence of the compounds, the concentration-response curve for the isoproterenol-induced increase in cAMP was shifted to the right with no apparent reduction in the maximal responses, indication that these compounds are competitive antagonists of β₁- and β₂-ARs. Using the Schild equations for multiple concentrations of the antagonist (ranolazine) or for a single concentration of the enantiomers, the K_{B} values for ranolazine and its S- and R-enantiomers were 9.7, 8.0, and >50 μM (for β₁-ARS) and 12.2, 9.0, and >50 μM (for β₂-ARs). In summary, ranolazine and its S-enantiomer are competitive antagonists of β₁ and β₂-ARS with the S-enantiomer having the highest binding affinities of all.

### Example 9

### Effect of the R- and S-Enantiomers of Ranolazine on I_{Kr}, I_{Ks}, and I_{Na}

The objective of this example is to demonstrate the effects of ranolazine's S- and R-enantiomers on I_{Kr}, I_{Ks}, and late I_{Na}. Whole cell currents were recorded from isolated canine left ventricular midmyocrdial cells at 37° C. I_{Kr} and I_{Ks} were recorded during standard pulse protocols. Action potentials recorded at basic cycle lengths (BCL) of 300 and 2000 ms were used as command waveforms during voltage claim to measure late I_{Na}. Enantiomeric effects were determined at concentrations of 3, 10, and 30 μM.

Late I_{Na} was evaluated at two voltages during the plateau and final repolarization of an action potential voltage claims. S- and R-ranolazine inhibited late I_{Na} in a concentration-dependent manner. Just as with racemic ranolazine, inhibition by the enantiomers was greatest at plateau potentials and during rapid stimulation. At a BCL of 300, half-inhibition (IC₅₀) of late I_{Na} by S-ranolazine and R-ranolazine occurred at 5±0.4 μM and 8±2.6 μM, respectively (R vs S, IC₅₀ for I_{Kr} was 10±2 μM for S-ranolazine and 28±4 μM for R-ranolazine (r vs S; p<0.05). I_{Ks} was reduced 27% by 30 μM S-ranolazine, whereas the same concentration of R-ranolazine reduced I_{Ks} by only 5% (p<0.004). In comparison, racemic ranolazine has been shown to have an IC₅₀ for late I_{Na} of 5 μM, an IC₅₀ for I_{Kr} of 11 μM, and an IC₅₀ value of >100 μM (see Zygmunt et al. (2002) Pacing Clin. Electrophysiol 25, II-626, Abstract). In conclusion R-ranolazine produces less I_{Kr} and I_{Ks} block and near equivalent inhibition of late I_{Na} to that produced by S-ranolazine or the racemic form of the drug.

### Example 10

### Effect of Ranolazine and the R- and S-Enantiomers of Ranolazine on LV MAPD

The objective of this example is to present the effects of ranolazine and the R- and S-enantiomers of ranolazine on left ventricular monophasic action potential duration (LV MAPD), regional LV dispersion of MAPD (apex-base) and arrhythmogenesis. Female rabbit isolated hearts were perfused with modified K-H solution and paced at a constant rate of 1 Hz after surgical-induced complete atrioventricular block. LV MAPs from the base and apex were measured using contact MAP electrodes. Ranolazine (1-100 μM) caused a similar concentration-dependent prolongation of the basal and apical MAPD₉₀, i.e. a 32±4% and 35±4% increases above control with an EC₅₀ of 4.3 and 4.8 μmol/L (n=7), respectively, without increasing regional MAPD dispersion or eliciting premature ventricular beats (PVBs) or ventricular tachycardia (VT). The enantiomers of ranolazine, R and S, prolonged MAPD₉₀ with a maximal increase of 22±5 and 28±4% (n=11 and 9, respectively).

### Materials

Ranolazine, (±)-N-(2,6-Dimethylphenyl)-(4[2-hydroxy-3-(2-methoxyphenoxy)propyl]-1-piperazine (Lot # E4-NE-002), ranolazine R-isomer (Lot # SAR-224-12-6) and S-isomer (Lot # SAR-224-24-11) were synthesized by the Bio-organic Chemistry Department of CV Therapeutics, Inc. (Palo Alto, Calif.). E-4031 (1[2-(6-methyl-2-pyridyl)ethyl]-4-methylsulfonylaminobenzoyl)piperidine, Lot # E-04) and ATX-II (anemonia sulcata, Lot # AT-04) were purchased from Alomone Labs (Jerusalem, Israel). Dimethylsulfoxide (100% DMSO; Sigma, St. Louis, Mo.) was used to prepare stock solutions of 40 mmol/L concentrations of ranolazine and its R and S isomers. ATX-II and E-4031 were dissolved in saline to prepare stock solutions at 10 μmol/L and 1 mmol/L, respectively. All solutions were stored at −4 to −20° C. and diluted in physiological saline before use. The final DMSO content in final solutions used to perfuse the hearts was <0.1%. Female rabbits (New Zealand White, 2.5-3.5 kg) were obtained from Western Oregon Rabbitry (Philomath, Oreg.). Animal use was reviewed and approved by the Institutional Animal Care and Use Committee of CV Therapeutics, Inc.

### Experimental Preparation

Each animal was sedated using 6 mg/kg xylazine i.m. and 40 mg/kg ketamine i.m. and then anesthetized by a “cocktail” (ketamine 15 mg/kg+xylazine 4 mg/kg in 1.5 mL saline) i.v. via the marginal ear vein. The thorax was quickly opened. The heart was excised and placed in a modified Krebs-Henseleit (K-H) solution at room temperature. The K-H solution contained (in mmol/L): NaCl 118, KCl 2.8, KH₂PO₄ 1.2, CaCl₂ 2.5, MgSO₄ 0.5, pyruvate 2.0, glucose 5.5, Na₂EDTA 0.57 and NaHCO₃ 25. The solution was continuously gassed with 95% O₂ and 5% CO₂, and its pH was adjusted to 7.4. The aorta was rapidly catheterized and the heart was perfused by the method of Langendorff with K-H solution warmed to 36-36.5° C. at a rate of 20 mL/min with a roller pump (Gilson Minipuls3, Middleton, Wis.). Perfusion pressure was measured (with a Biopac MP 150 pressure transducer, Goleta, Calif.) from a side port of the aortic catheter. To facilitate exit of fluid from the chamber of the left ventricle (LV), the leaflets of the mitral valve were trimmed with fine spring-handled scissors. The right atrial wall was partially removed for AV node ablation to block AV conduction.

Complete AV block was induced by surgical ablation (heated) of AV nodal area. The spontaneous ventricular rate (i.e., the ventricular escape rhythm) was a few beats per minute after successful AV nodal ablation. A bipolar Teflon-coated electrode was placed on the right ventricular septum to pace the heart. Electrical stimuli 3 msec in width and 3-fold threshold amplitude were delivered to the pacing electrode at a frequency of 1 Hz using a Grass S48 stimulator (W. Warwick, R.I.).

After initiation of ventricular pacing, a 30-40 min delay was allowed for heart rhythm and perfusion pressure to achieve a steady state, an essential experimental condition for recording a good quality monophasic action potential (MAP). The total duration of the experimental protocol was limited to 2.5 h, the time during which the preparation exhibited good stability.

### Signal Recording and Processing

Monophasic action potentials (MAP) and ECG electrodes from Harvard Apparatus Inc. (Holliston, Mass.) were used to record left ventricular MAPs and bipolar ECG. Two MAP electrodes were placed on the epicardial ventricular free wall below the level of atrial-ventricular valves to record basal MAP and apex to record apical MAP signals, respectively. MAP electrodes were pressure-contact Ag—AgCl electrodes attached to a circular holder with springs to maintain the electrodes' contact with the LV epicardial surface. Electrode signals were amplified and displayed on a computer monitor for visual monitoring throughout the experiments. To ensure that each response to a drug(s) had achieved a steady state before a drug concentration was changed, the MAP duration (from onset of depolarization to 100% repolarization) was measured using an on-screen caliper throughout each drug infusion period. Signals were saved on a computer hard disk for subsequent analysis. Bipolar electrocardiogram (ECG) was generated using an isolated-heart ECG apparatus (Harvard Apparatus, Holliston, Mass.) attached to Biopac amplifier system. Coronary perfusion pressure was measured using a pressure transducer (Biopac or PowerLab pressure measuring system). MAPs, ECGs, and coronary perfusion pressure (CPP) signals were appropriately amplified, filtered, sampled, and digitized in real time (using a Biopac MP 150, Goleta, Calif.), and displayed on a computer screen. All signals were saved on a computer hard disk for subsequent analysis.

Original MAP profiles of 3-5 consecutive beats were superimposed to get one average signal using computer software (Biopac, Goleta, Calif.) and then transferred into Microsoft Excel software and plotted to measure the duration of the MAP at the level at which repolarization is 90% completed (MAPD₉₀).

### Exclusion Criteria

Any of the following problems during the equilibration period were cause for excluding a preparation from this study: (1) unstable coronary perfusion pressure; (2) persistent premature ventricular complexes (PVBs) or ventricular tachycardia after AV nodal ablation; (3) macroscopic anatomical damage to the heart; or (4) MAP signal instability. Approximately 5% of all preparations were excluded.

### Statistical Analysis

All data are reported as means±SEM. Concentration-response curves were analyzed using Prism Version 3.0 (GraphPad, San Diego, Calif.). To compare values of measurements obtained from the same heart before and after a drug treatment, repeated measures one-way analysis of variance (ANOVA) was used (SigmaStat, IL). When the ANOVA revealed a significant difference among values, the Student-Newman-Keuls test and Student t-test were applied to determine which pairs of group means were significantly different. A significant difference between 2 group means was defined as p<0.05. The effects of drugs were sometimes calculated as percent change from control in order to facilitate the interpretation of responses to drugs in different hearts.

Testing with Ranolazine and R- and S-Enantiomers

Increasing concentrations of ranolazine and its R- and S-enantiomers (concentration range for each drug was 0.1-100 μmol/L) were infused into the rabbit hearts in a cumulative manner, allowing 7-15 min between changes of ranolazine concentration to attain steady state effect to construct the concentration-MAPD₉₀ response curves. All hearts were paced at 1 Hz throughout the experimental procedure. The maximum MAP prolongation was measured during the infusion of each concentration of drugs.

LV basal and apical MAPs were recorded simultaneously throughout the experiments. MAP durations, from the Base and Apex of the heart, at the level of repolarization that is 90% completed (MAPD₉₀) were measured to compare the MAP prolongation, regional LV MAPD dispersion caused by each drug.

Ranolazine prolonged MAPD₉₀ recorded from the rabbit left ventricle paced at 1 Hz in a concentration-dependent manner. The maximal increases of basal and apical MAPD₉₀ were similar as 32.2±4.2% and 35.2±4.1% (n=7, p<0.01), respectively, above control (no drug) at ranolazine concentrations of 30-100 μmol/L. The estimated potencies (EC₅₀ values) for ranolazine to prolong LV MAPD₉₀ (Base and Apex) were 4.3 and 4.8 μmol/L, respectively. Ranolazine did not induce any EADs, PVBs or VT at any concentration.

The enantiomers of ranolazine, R and S, also prolonged MAPD₉₀ with a maximal increase of 22±5 and 28±4%, respectively. The EC₅₀, for the R and S isomers to prolong LV MAPD₉₀ were 6.4 and 5.9 μmol/L, respectively, indicating that the S isomer is more potent than the R isomer.

We claim:

1. A method for enhancing endogenous insulin levels in a patient in need thereof which method comprises administering to the patient an insulin secretion-enhancing amount of ranolazine as the racemate or the R- or S-enantiomer of ranolazine.

2. The method of claim 1, wherein the patient is insulin-responsive and insulin secretion-deficient.

3. The method of claim 2, wherein the patient is pre-diabetic or otherwise disposed to diabetes mellitus.

4. The method of claim 2, wherein the patient suffers from type II diabetes mellitus.

5. The method of claim 1, wherein the insulin secretion-enhancing amount of ranolazine is in the form of the racemate.

6. The method of claim 1, wherein the insulin secretion-enhancing amount of ranolazine is in the form of the R- or S-enantiomer of ranolazine.

7. The method of claim 6, wherein the insulin secretion-enhancing amount of ranolazine is in the form of the R-enantiomer of ranolazine.

8. A method for reducing the amount and/or frequency of insulin administration to a patient, which method comprises administering to the patient an insulin secretion-enhancing amount of ranolazine as the racemate or the R- or S-enantiomer of ranolazine.

9. The method of claim 8, wherein the insulin secretion-enhancing amount of ranolazine is in the form of the racemate.

10. The method of claim 8, wherein the insulin secretion-enhancing amount of ranolazine is in the form of the R-enantiomer of ranolazine.

11. A method for treating a diabetic patient, which method comprises administering to the patient an insulin secretion-enhancing amount of ranolazine as the racemate or the R- or S-enantiomer of ranolazine in combination with at least one anti-diabetic agent.

12. The method of claim 11, wherein the insulin secretion-enhancing amount of ranolazine is in the form of the racemate.

13. The method of claim 11, wherein the insulin secretion-enhancing amount of ranolazine is in the form of the R-enantiomer of ranolazine.

14. A method for maintaining effectiveness of anti-diabetic therapy in a patient, wherein the method comprises administering to the patient an insulin secretion-enhancing amount of ranolazine as the racemate or the R- or S-enantiomer of ranolazine in combination with the anti-diabetic.

15. The method of claim 14, wherein the insulin secretion-enhancing amount of ranolazine is in the form of the racemate.

16. The method of claim 14, wherein the insulin secretion-enhancing amount of ranolazine is in the form of the R-enantiomer of ranolazine.

17. A composition comprising an insulin secretion-enhancing amount of ranolazine as the racemate or the R- or S-enantiomer of ranolazine and at least one anti-diabetic agent.

18. The composition of claim 17, wherein the insulin secretion-enhancing amount of ranolazine is in the form of the racemate.

19. The composition of claim 17, wherein the insulin secretion-enhancing amount of ranolazine is in the form of the R-enantiomer of ranolazine.

20. The composition of claim 17, wherein said anti-diabetic agent is selected from the group consisting of sulfonylureas, DPP-IV inhibitors, biguanides, thiazolidindiones, alpha-glucosidase inhibitors, incretin mimetics, PPAR gamma modulators, dual PPARalpha/gamma agonists, RXR modulators, SGLT2 inhibitors, aP2 inhibitors, insulin sensitizers, PTP-IB inhibitors, GSK-3 inhibitors, DP4 inhibitors, insulin sensitizers, insulin, meglitinide, PTP1B inhibitors, glycogen phosphorylase inhibitors, glucose-6-phosphatase inhibitor, and amylin analogs.

21. The composition of claim 20, wherein said anti-diabetic agent is selected from the group consisting of metformin, phenformin, buformin, chlorpropamide, glisoxepid, glyburide, acetohexamide, chlorpropamide, glibornuride, tolbutamide, tolazamide, glipizide, glimepiride, gliclazide, gliquidone, glyhexamide, phenbentamide, tolcyclamide, troglitazone, pioglitazone, rosiglitazone, miglitol, acarbose, exenatide, vildagliptin, sitagliptin, repaglinide, pramlintide, and nateglinide.

22. The method of claim 11, wherein said anti-diabetic agent is selected from the group consisting of sulfonylureas, DPP-IV inhibitors, biguanides, thiazolidindiones, alpha-glucosidase inhibitors, incretin mimetics, PPAR gamma modulators, dual PPARalpha/gamma agonists, RXR modulators, SGLT2 inhibitors, aP2 inhibitors, insulin sensitizers, PTP-IB inhibitors, GSK-3 inhibitors, DP4 inhibitors, insulin sensitizers, insulin, meglitinide, PTP1B inhibitors, glycogen phosphorylase inhibitors, glucose-6-phosphatase inhibitor, and amylin analogs.

23. The method of claim 22, wherein said anti-diabetic agent is selected from the group consisting of metformin, phenformin, buformin, chlorpropamide, glisoxepid, glyburide, acetohexamide, chlorpropamide, glibornuride, tolbutamide, tolazamide, glipizide, glimepiride, gliclazide, gliquidone, glyhexamide, phenbentamide, tolcyclamide, troglitazone, pioglitazone, rosiglitazone, miglitol, acarbose, exenatide, vildagliptin, sitagliptin, repaglinide, pramlintide, and nateglinide.

24. The method of claim 14, wherein said anti-diabetic agent is selected from the group consisting of sulfonylureas, DPP-IV inhibitors, biguanides, thiazolidindiones, alpha-glucosidase inhibitors, incretin mimetics, PPAR gamma modulators, dual PPARalpha/gamma agonists, RXR modulators, SGLT2 inhibitors, aP2 inhibitors, insulin sensitizers, PTP-IB inhibitors, GSK-3 inhibitors, DP4 inhibitors, insulin sensitizers, insulin, meglitinide, PTP1B inhibitors, glycogen phosphorylase inhibitors, glucose-6-phosphatase inhibitor, and amylin analogs.

25. The method of claim 24, wherein said anti-diabetic agent is selected from the group consisting of metformin, phenformin, buformin, chlorpropamide, glisoxepid, glyburide, acetohexamide, chlorpropamide, glibornuride, tolbutamide, tolazamide, glipizide, glimepiride, gliclazide, gliquidone, glyhexamide, phenbentamide, tolcyclamide, troglitazone, pioglitazone, rosiglitazone, miglitol, acarbose, exenatide, vildagliptin, sitagliptin, repaglinide, pramlintide, and nateglinide.

26. A method for treating a patient suffering from one or more cardiovascular diseases which method reduces adverse events and/or drug-drug interactions while enhancing endogenous insulin levels, comprising administering the R-enantiomer of ranolazine to these patients.

27. The method of claim 26 wherein administration is oral.

28. The method of claim 27 wherein the R-enantiomer of ranolazine is administered as a sustained release formulation.

29. The method of claim 27, wherein the R-enantiomer of ranolazine is administered as an immediate release formulation.

30. The method of claim 1 wherein the patient is treated with the R-enantiomer of ranolazine without testing the patient to determine if there is a dysfunction of the CYP2D6 enzyme.

31. The method of claim 26, wherein the at least one cardiovascular disease or cardiovascular disease symptom is selected from heart failure, including congestive heart failure, acute heart failure, myocardial infarction, and the like, arrhythmias including treatment of supra ventricular tachycardias such as atrial fibrillation, atrial flutter, AV nodal reentrant tachycardia, atrial tachycardia, and the ventricular tachycardias (VTs), including idiopathic ventricular tachycardia, ventricular fibrillation, pre-excitation syndrome, and Torsade de Pointes (TdP), angina, including exercise-induced angina, variant angina, stable angina, unstable angina, acute coronary syndrome, and the like, and peripheral artery disease, including intermittent claudication.

32. The method of claim 26, wherein the patient is insulin-responsive and insulin secretion-deficient.

33. The method of claim 32, wherein the patient is pre-diabetic or otherwise disposed to diabetes mellitus.

34. The method of claim 32, wherein the patient suffers from type II diabetes mellitus.

35. A pharmaceutical composition comprising a therapeutically effective amount of the R-enantiomer of ranolazine or a pharmaceutically acceptable salt, ester, prodrug, or hydrate thereof.

36. A method for treating a patient suffering from one or more cardiovascular diseases which method reduces adverse events, comprising administering the S-enantiomer of ranolazine to these patients.

37. The method of claim 36 wherein administration is oral.

38. The method of claim 37 wherein the S-enantiomer of ranolazine is administered as a sustained release formulation.

39. The method of claim 37, wherein the S-enantiomer of ranolazine is administered as an immediate release formulation.

40. The method of claim 36, wherein the at least one cardiovascular disease or cardiovascular disease symptom is selected from heart failure, including congestive heart failure, acute heart failure, myocardial infarction, and the like, arrhythmias including treatment of supra ventricular tachycardias such as atrial fibrillation, atrial flutter, AV nodal reentrant tachycardia, atrial tachycardia, and the ventricular tachycardias (VTs), including idiopathic ventricular tachycardia, ventricular fibrillation, pre-excitation syndrome, and Torsade de Pointes (TdP), angina, including exercise-induced angina, variant angina, stable angina, unstable angina, acute coronary syndrome, and the like, and peripheral artery disease, including intermittent claudication.

41. The method of claim 36, wherein the patient is diabetic, pre-diabetic, or otherwise disposed to diabetes mellitus further comprising administering a therapeutically effective amount of the R-enantiomer of ranolazine in an amount that is different that the amount of the S-enantiomer to be administered.

42. The method of claim 41, wherein the patient is pre-diabetic or otherwise disposed to diabetes mellitus.

43. The method of claim 41, wherein the patient suffers from type II diabetes mellitus.

44. A pharmaceutical composition comprising a therapeutically effective amount of the S-enantiomer of ranolazine or a pharmaceutically acceptable salt, ester, prodrug, or hydrate thereof.

45. A pharmaceutical composition comprising therapeutically effective non-equal amounts of the R-enantiomer and the S-enantiomer of ranolazine or pharmaceutically acceptable salts, esters, prodrugs, or hydrates thereof.
